(19) United States
(12) Patent Application Publication
(10) Pub. No.: US 2011/0034686 A1
(43) Pub. Date: Feb. 10, 2011

(54) Process for Isolation and Purification of Geldanamycin

(76) Inventors: Nitin Sopanrao Patil, Karnataka (IN); Rakesh Bhaiyyaram Mendhe, Maharashtra (IN); Anantrao Phatale Amit, Maharashtra (IN); Prakash Khedkar Anand, Banglore (IN)

Correspondence Address: DLA PIPER LLP (US), 4365 EXECUTIVE DRIVE, SUITE 1100, SAN DIEGO, CA, 92121-2133, US

(21) Appl. No.: 12/988,802

(22) PCT Filed: Jun. 6, 2008
(86) PCT No.: PCT/IN08/00355

§ 371 Date: Oct. 20, 2010

(30) Foreign Application Priority Data
Apr. 21, 2008 (IN) 00967/CHE/2008

(51) Int. Cl. C07D 225/04

(52) U.S. Cl. 540/461

(57) ABSTRACT

The present invention is successful in providing a suitable process for the isolation and purification of geldanamycin. The process provided in the instant invention is easy to scale-up, industrially safe and will give high yield and productivity.

### FIELD OF THE INVENTION

This invention relates to a process for isolation and purification of geldanamycin.

### BACKGROUND AND PRIOR ART OF THE INVENTION

A compound, (4E,6Z,8S,9S,10E,12S,13R,14S,16R)-13-hydroxy-8,14, 19-trimethoxy-4,10,12,16-tetramethyl-3,20,22-trioxo-2-azabicyclo[16.3.1] docosa-1(21), 4,6,10,18-pentaen-9-yl carbamate also known as Geldanamycin is disclosed by U.S. Pat. No. 3,595,955. Geldanamycin is a natural product of the filamentous bacterium Streptoinyces hygroscopicus (J. Antibiotics, 23, 1970, 442-447). Geldanamycin is considered to be a mixture of two unresolved chemical compounds with the formulae C29H40N209 and C29H42N209. Recognized as having antiprotozoic activity, this antibiotic was also known to have high activity against human epidermoid carcinoma cells (see U.S. Pat. No. 3,595,955 and J. Antibiotics 24, 1976, 1182-1188). Subsequently, geldanamycin antitumor activity has been demonstrated against 60 cell lines (see Cancer Chemother. Pharmacol, 36 (4), 1995, 305-315). Furthermore, geldanamycin has been shown to selectively inhibit heat shock protein 90 (hsp90), a molecular chaperone responsible for protein folding and maturation in vivo and which has been found at higher levels in cancerous cells than in normal cells (see J. Biol. Chem., 275 (41), 2000, 31682-31688 and Exp. Cell Res., 262 (1), 2001, 59-68).

Several derivatives of geldanamycin have been shown to be effective as anticancer agents (hsp90 inhibitors) and are under various phases of clinical trials, e. g., 17-Allylamino-17-demethoxygeldanamycin (see Cancer Chemother. Pharmacol, 42 (4), 1998, 273-279), 17-Dimethyaminoethylamino-demethoxygeldanamycin (Curr. Cancer Drug Targets, 3 (5), 2003, 297-300), 17-(3-(4-Maleimidobutyrcarboxamido) propylamino)-demethoxygeldanamycin (see Cancer Research, 64 (4), 2004, 1460-1467), 17-[2-(pyrrolidin-1-yl)ethyl]amino-17-demthodygeldanamycin (see Bioorg. Med. Chem, 12 (20) 5317-5329). Few reduction product of derivatives of geldanamycin shows selective toxicity to tumor cells, e. g., reduced product of 17-Allylamino-17-demethoxygeldanamycin and reduced product of 17-Dimethyaminoethylamino-demethoxygeldanamycin (see J. Natl. Cancer Inst. 91 (22), 1999, 1940-1949).

U.S. Pat. 3,595,955 discloses a process for the isolation and purification of geldanamycin. This process uses filtration for removal of cell mass, silica gel chromatography for purification, crystallization through chloroform above ambient temperature. Filtration is a tedious and time consuming unit operation. Silica gel requires very high amount of solvents and disposal of used silica gel. Crystallization through hot chloroform is unsafe. WO/2003/072794 also discloses a process for the isolation and purification of geldanamycin comprising of pH adjustment, filtration to remove the cell mass below ambient temperatures, crystallization. Here, filtration is a time consuming and tedious unit operation and also, this process gives maximum yield of nearly 47%.

It is, therefore, an object of the present invention to provide an improved process for isolating and purifying geldanamycin, which will be easy to scale-up, industrially safe and will give high yield and productivity.

### OBJECTS OF THE INVENTION

The main object of the present invention is to provide a process for the purification of geldanamycin.

Yet another object of the present invention is to provide a purified geldanamycin.

### STATEMENT OF THE INVENTION

Accordingly, the present invention is in relation to a process for the purification of geldanamycin, said process comprising steps of: extraction of a fermentation broth containing geldanamycin with an organic solvent or a mixture of organic solvents; adsorption of extracted product of step (a) on a solid support; washing of the product-containing solid support of step (b) with an organic solvent or a mixture of organic solvents or a mixture of an organic solvent and water; further washing the product-containing solid support from step (c) with an organic solvent or mixture of organic solvents to cause product elution; crystallizing the eluted product from step (d) followed by filtration; purification of the filtered solids from step (e) by making a suspension in an organic solvent or mixture of organic solvents and filtration; optional repetition of step (f) and drying.

### DETAILED DESCRIPTION OF THE INVENTION

The present invention is in relation to a process for the purification of geldanamycin, said process comprising steps of:
a. extraction of a fermentation broth containing geldanamycin with an organic solvent or a mixture of organic solvents;
b. adsorption of extracted product of step (a) on a solid support;
c. washing of the product-containing solid support of step (b) with an organic solvent or a mixture of organic solvents or a mixture of an organic solvent and water;
d. further washing the product-containing solid support from step (c) with an organic solvent or mixture of organic solvents to cause product elution;
e. crystallizing the eluted product from step (d) followed by filtration;
f. purification of the filtered solids from step (e) by making a suspension in an organic solvent or mixture of organic solvents and filtration;
g. optional repetition of step (f) and
h. drying

In another embodiment of the present invention the organic solvents are selected from a group comprising alcohols, alkanes, chlorinated alkanes, ketones, acetates or ethers.

In yet another embodiment of the present invention said alcohols are selected from a group comprising methanol, ethanol, propanol or butanol.

In still another embodiment of the present invention said alkanes are selected from a group comprising hexane, heptane or pentane.

In still another embodiment of the present invention said chlorinated alkanes are selected from a group comprising methylene di chloride, ethylene di chloride or chloroform.

In still another embodiment of the present invention said ketones are selected from a group comprising acetone, methyl ethyl ketone or methyl isobutyl ketone.

In still another embodiment of the present invention said acetates are selected from a group comprising ethyl acetate, propyl acetate or butyl acetate.

In still another embodiment of the present invention said ethers are selected from a group comprising diethyl ether, petroleum ether or diisopropyl ether.

In still another embodiment of the present invention the solid support for adsorption is selected from a group comprising diatomaceous earth, celite, charcoal or polystyrene-divinylbenzene.

In still another embodiment of the present invention the crystallization is done by the addition of anti solvents.

In still another embodiment of the present invention said anti solvents are selected from a group comprising acetone, methyl ethyl ketone, acetonitrile, pentane, hexane, heptane, ethyl acetate, propyl acetate, butyl acetate, methanol, ethanol, propanol, butanol, diethyl ether, methyl tert-butyl ether, diisopropyl ether, petroleum ether or mixture thereof.

In still another embodiment of the present invention purification of the filtered solids from step (e) comprises;
a. dissolving impure geldanamycin in an organic solvent;
b. passing solution of impure geldanamycin through a bed of adsorbent preferably alumina and collecting the flow-through;
c. optionally washing the bed of adsorbent from step (b) with organic solvent or a mixture of organic solvents and collecting the elute;
d. optionally combining the flow-through from step (b) and elute from step (c);
e. concentrating the combined product layer from step (d) so that the concentrate weight becomes about 10 times the weight of the product;
f. crystallizing geldanamycin optionally cooling to the temperature less than 15° C.;
g. filtrating the crystals obtained in step (f) and
h. drying

In still another embodiment of the present invention said organic solvents are selected from a group comprising alcohols, alkanes, chlorinated alkanes, ketones, acetates or ethers.

In still another embodiment of the present invention said alcohols are selected from a group comprising methanol, ethanol, propanol or butanol.

In still another embodiment of the present invention said alkanes are selected from a group comprising hexane, heptane or pentane.

In still another embodiment of the present invention said chlorinated alkanes are selected from a group comprising methylene di chloride, ethylene di chloride or chloroform.

In still another embodiment of the present invention said ketones are selected from a group comprising acetone, methyl ethyl ketone or methyl isobutyl ketone.

In still another embodiment of the present invention said acetates are selecting from ethyl acetate, propyl acetate or butyl acetate.

In still another embodiment of the present invention said ethers are selecting from diethyl ether, petroleum ether or diisopropyl ether.

The instant invention relates to a process for recovery and purification of geldanamycin, which comprises of:
a) extraction of fermentation broth containing geldanamycin with an organic solvent or mixture of organic solvents,
b) adsorption of extracted product from step (a) on a solid support,
c) washing of the product-containing solid support from step (b) with an organic solvent or mixture of organic solvents or mixture of organic solvent and water,
d) further washing of product-containing solid support from step (c) with an organic solvent or mixture of organic solvents to cause product elution,
e) crystallization of the eluted product from step (d) followed by filtration,
f) purification of the filtered solids from step (e) by making its suspension in an organic solvent or mixture of organic solvents and filtration,
g) optional repetition of step (f) and
h) drying

The instant invention also relates to a process of purification of geldanamycin comprising of,
a. dissolving impure geldanamycin in an organic solvent or mixture thereof,
b. passing solution of impure geldanamycin through a bed of adsorbent preferably alumina and collecting the flow-through,
c. optionally washing the bed of adsorbent from step (b) with organic solvent or mixture of organic solvents and collecting the elute,
d. optionally combining the flow-through from step (b) and elute from step (c),
e. concentration of the combined product layer from step (d) so that the concentrate weight becomes about 10 times the weight of the product,
f. crystallization of geldanamycin optionally cooling to the temperature less than 15° C.,
g. filtration of crystals obtained in step (f), and
h. drying

The thus obtained product is of acceptable quality.

As mentioned earlier, the instant invention relates to a process for the isolation and purification of geldanamycin. The process of the instant invention comprises of:
a) extraction of fermentation broth containing geldanamycin with an organic solvent or mixture of organic solvents,
b) adsorption of extracted product from step (a) on a solid support,
c) washing of the product-containing solid support from step (b) with an organic solvent or mixture of organic solvents or mixture of organic solvent and water,
d) further washing of product-containing solid support from step (c) with an organic solvent or mixture of organic solvents to cause product elution,
e) crystallization of the eluted product from step (d) followed by filtration,
f) purification of the filtered solids from step (e) by making its suspension in an organic solvent or mixture of organic solvents and filtration,
g) optional repetition of step (f) and
h) drying

The geldanamycin of the present invention can be produced by fermentation or synthesized. The broth obtained by fermentation can be extracted using organic solvent. The broth may be extracted directly or after processing to yield crude material in solid, semisolid or liquid form. The organic solvent may be selected from alkanols, e.g., methanol, ethanol, propanol, the butanol, and the like; chlorinated alkanes, e. g., methylene chloride, chloroform, ethylene dichloride, and the like; ketones, e.g., acetone, methyl ethyl ketone, and the like; acetates, e.g., ethyl acetate, propyl acetate, butyl acetate and the like or mixtures thereof. The aqueous and the organic layers may be separated by known processes including centrifugation or filtration.

The solid support may be selected from organic or inorganic supports or mixtures thereof. Preferably, the support may be selected from inert materials like diatomaceous earth, celite, charcoal, polystyrenedivinylbenzene and the like. The product in the organic extract can be adsorbed onto the solid support by mixing the two and evaporating the solvent. The evaporation of solvent can be affected by methods known per se. The evaporation can be affected by vaporization of the solvent. The vaporization of the solvent can be carried out by heating without or with reduced pressure.

The solid support containing adsorbed product can be washed with organic solvent and then eluted with organic solvent. The organic solvent for washing and elution may be independently selected from water, alkanols, e.g., methanol, ethanol, isopropanol, the butanol, and the like; chlorinated alkanes, e. g., methylene chloride, chloroform, ethylene dichloride, and the like; alkanes, e. g., heptane, hexane, pentane and the like; ketones, e.g., acetone, methyl ethyl ketone, and the like; acetates, e.g., ethyl acetate, propyl acetate, butyl acetate and the like and ethers e.g., diethyl ether, petroleum ether and the like or mixtures thereof. Preferably, the solvent may be selected from water, methanol, ethanol, isopropyl alcohol, acetone, acetonitrile, and methylene dichloride or mixture thereof.

The product from the elute can be then crystallized. The crystallization may be carried out by known methods including evaporation of solvent, addition of an anti-solvent, reducing the temperature or combination thereof. The evaporation of solvents can be affected by methods known per se. The evaporation can be affected by vaporization of the solvent. The vaporization of the solvent can be carried out by heating without or with reduced pressure. The anti-solvent may be selected from acetone, methyl ethyl ketone, acetonitrile, pentane, hexane, heptane, ethyl acetate, propyl acetate, butyl acetate, methanol, ethanol, propanol, butanol, diethyl ether, methyl tert-butyl ether, diisopropyl ether, petroleum ether or mixture thereof. The crystallized product may be isolated by filtration or centrifugation.

The crystals can be further purified by making its suspension in organic solvent. The solvent can be selected from alkanols, e.g., methanol, ethanol, isopropanol, the butanol, and the like; chlorinated alkanes, e. g., methylene chloride, chloroform, ethylene dichloride, and the like; ketones, e.g., acetone, methyl ethyl ketone, and the like; acetates, e.g., ethyl acetate, butyl acetate and the like and ethers e.g., diethyl ether and the like and mixtures thereof. Preferably the crystals are suspended in solvent repeatedly to achieve the acceptable quality product. The product from the suspension can be filtered and dried.

In particular, the process of the instant invention comprises:
a) extraction of fermentation broth containing geldanamycin with an organic solvent or mixture of organic solvents,
b) adsorption of extracted product from step (a) on a solid support,
c) washing of the product-containing solid support from step (b) with an organic solvent or mixture of organic solvents or mixture of organic solvent and water,
d) further washing of product-containing solid support from step (c) with an organic solvent or mixture of organic solvents to cause product elution,
e) crystallization of the eluted product from step (d) followed by filtration,
f) purification of the filtered solids from step (e) by making its suspension in an organic solvent or mixture of organic solvents and filtration,
g) optional repetition of step (f) and
h) drying

As mentioned earlier, the instant invention also relates to a process of purification of geldanamycin. The process of the instant invention comprises of:
a. dissolving impure geldanamycin in an organic solvent,
b. passing solution of impure geldanamycin through a bed of adsorbent preferably alumina and collecting the flow-through,
c. optionally washing the bed of adsorbent from step (b) with organic solvent
or mixture of organic solvents and collecting the elute,
d. optionally combining the flow-through from step (b) and elute from step (c),
e. concentration of the combined product layer from step (d) so that the
concentrate weight becomes about 10 times the weight of the product,
f. crystallization of geldanamycin from the concentrate obtained in step (e) optionally cooling to the temperature less than 15° C.
g. filtration of crystals obtained in step (1), and
h. drying

The thus obtained product is of acceptable quality.

The impure geldanamycin can be obtained by fermentation or chemical reactions. The impure geldanamycin can be dissolved in organic solvent. The organic solvent may be selected from alkanols, e.g., methanol, ethanol, propanol, the butanol, and the like; chlorinated alkanes, e. g., methylene chloride, chloroform, ethylene dichloride, and the like; ketones, e.g., acetone, methyl ethyl ketone, and the like; acetates, e.g., ethyl acetate, butyl acetate and the like and ethers and mixtures thereof.

The geldanamycin solution can be passed through a bed of adsorbent. The adsorbent can be organic or inorganic solid supports including alumina, silica gel, charcoal, polystyrene divinyl benzene resin and the like. The flow-through can be collected. The product bound to the adsorbent may be further eluted using organic solvent. The solvent can be selected from alkanols, e.g., methanol, ethanol, isopropyl, the butanol, and the like; chlorinated alkanes, e. g., methylene chloride, chloroform, ethylene dichloride, and the like; ketones, e.g., acetone, methyl ethyl ketone, and the like; acetates, e.g., ethyl acetate, butyl acetate and the like and mixtures thereof.

The flow-through and the pure product containing elute can be pooled. The product from pooled layer can be crystallization. The crystallization may be carried out by known methods including evaporation of solvent, addition of an anti-solvent, reducing the temperature or combination thereof. The evaporation of solvents can be affected by methods known per se. The evaporation can be affected by vaporization of the solvent.

The vaporization of the solvent can be carried out by heating without or with reduced pressure. The anti-solvent may be selected from acetone, methyl ethyl ketone, acetonitrile, pentane, hexane, heptane, ethyl acetate, propyl acetate, butyl acetate, methanol, ethanol, propanol, butanol, diethyl ether, methyl tert-butyl ether or mixture thereof. The crystallized product may be isolated by filtration or centrifugation.

The crystals can be further purified by suspending it in organic solvent. The solvent can be selected from alkanols, e.g., methanol, ethanol, isopropanol, the butanol, and the like; chlorinated alkanes, e. g., methylene chloride, chloroform, ethylene dichloride, and the like; ketones, e.g., acetone, methyl ethyl ketone, and the like; acetates, e.g., ethyl acetate, butyl acetate and the like and ethers e.g., diethyl ether and the like and mixtures thereof The product from the suspension can be filtered and dried.

In particular, the process of the instant invention comprises:
a. dissolving impure geldanamycin in an organic solvent,
b. passing solution of impure geldanamycin through a bed of adsorbent
Preferably alumina and collecting the flow-through,
c. optionally washing the bed of adsorbent from step (b) with organic solvent
Or mixture of organic solvents and collecting the elute,
d. optionally combining the flow-through from step (b) and elute from step (c),
e. concentration of the combined product layer from step (d) so that the
concentrate weight becomes about 10 times the weight of the product,
f. crystallization of geldanamycin optionally cooling to the temperature less
than 15° C.
g. filtration of crystals obtained in step (f), and
h. drying

The thus obtained product is of acceptable quality.

The technology of the instant Application is further elaborated with the help of following examples. However, the examples should not be construed to limit the scope of the invention.

### EXAMPLE 1

### Isolation and Purification of Geldanamycin

2450 kg of the fermentation broth was extracted thrice with mixture of acetone and ethyl acetate. The pooled extract showed 8.3 kg of product was mixed with celite. The mixture was concentrated under vacuum to obtain slurry. The slurry was mixed with methanol: water (60:40 v/v) mixture. The mixture was further concentrated. The mixture was filtered. The filtered solids were washed with methanol and water mixture (60:40 v/v). The solids were further washed with methanol. The product from the solids was then eluted using methylene dichloride: methanol mixture (80:20 v/v). The elute was concentrated. The mixture was cooled below 10° C. to cause crystallization. The crystals were then filtered. The crystals were washed with methanol dried to obtain 9.6 kg of geldanamycin powder. This powder was further mixed with methylene dichloride: diethyl ether mixture (60:40 v/v). The mixture was stirred and filtered. The filtered solids were further suspended in methylene dichloride: diethyl ether mixture (60:40 v/v). The mixture was stirred and filtered. These filtered solids were further suspended in acetone and filtered. The filtered solids were suspended in methylene dichloride: diethyl ether mixture (60:40 v/v). The suspension was filtered to get pure crystals. These crystals were dried to obtain 6.31 kg of final crystals.

### EXAMPLE 2

### Purification of Geldanamycin

300g of impure geldanamycin (chromatographic purity 96.7%) was dissolved in 12 L of methylene dichloride: methanol (60:40) mixture. The solution was passed through the bed of alumina under gravity. The flow-through was collected. The product from the bed is further eluted using methylene dichloride: methanol mixture (60:40). The flow-trough is concentrated below 40° C. so that the concentrate weight became 10 times the weight of the product. The concentrate is kept for crystallization below 10° C. for 2 h. The crystals are filtered and dried. The dried crystals showed chromatographic purity of 98.4%.

### EXAMPLE 3

### Purification of Geldanamycin

354 g of impure geldanamycin (chromatographic purity 96.9%) was dissolved in 12 L of methylene dichloride: methanol (60:40) mixture. The solution was passed through the bed of alumina under gravity. The flow-through was collected. The product from the bed is further eluted using methylene di chloride: methanol mixture (60:40). The flow-trough is concentrated below 40° C. so that the concentrate weight became 10 times the weight of the product. The concentrate is kept for crystallization below 10° C. for 2 h. The crystals are filtered and dried. The dried crystals showed chromatographic purity of 98.5%

1-19. (canceled)

20. A process for the purification of geldanamycin, said process comprising steps of
a. extraction of a fermentation broth containing geldanamycin with an organic solvent or a mixture of organic solvents;
b. adsorption of extracted product of step (a) on a solid support;
c. washing of the product-containing solid support of step (b) with an organic solvent or a mixture of organic solvents or a mixture of an organic solvent and water;
d. further washing the product-containing solid support from step (c) with an organic solvent or mixture of organic solvents to cause product elution;
e. crystallizing the eluted product from step (d) followed by filtration;
f. purification of the filtered solids from step (e) by making a suspension in an organic solvent or mixture of organic solvents and filtration;
g. optional repetition of step (f) and
h. drying

21. The process as claimed in claim 20, wherein the organic solvents are selected from a group comprising alcohols, alkanes, chlorinated alkanes, ketones, acetates or ethers.

22. The process as claimed in claim 21, wherein said alcohols are selected from a group comprising methanol, ethanol, propanol or butanol.

23. The process as claimed in claim 21, wherein said alkanes are selected from a group comprising hexane, heptane or pentane.

24. The process as claimed in claim 21, wherein said chlorinated alkanes are selected from a group comprising methylene di chloride, ethylene di chloride or chloroform.

25. The process as claimed in claim 21, wherein said ketones are selected from a group comprising acetone, methyl ethyl ketone or methyl isobutyl ketone.

26. The process as claimed in claim 21, wherein said acetates are selected from a group comprising ethyl acetate, propyl acetate or butyl acetate.

27. The process as claimed in claim 21, wherein said ethers are selected from a group comprising diethyl ether, petroleum ether or diisopropyl ether.

28. The process as claimed in claim 20, wherein the solid support for adsorption is selected from a group comprising diatomaceous earth, celite, charcoal or polystyrene-divinylbenzene.

29. The process as claimed in claim 20, wherein the crystallization is done by the addition of anti solvents, wherein the anti solvents are selected from a group comprising acetone, methyl ethyl ketone, acetonitrile, pentane, hexane, heptane, ethyl acetate, propyl acetate, butyl acetate, methanol, ethanol, propanol, butanol, diethyl ether, methyl tert-butyl ether, diisopropyl ether, petroleum ether or mixture thereof.

30. The process as claimed in claim 20, wherein purification of the filtered solids from step (e) comprises;
a. dissolving impure geldanamycin in an organic solvent;
b. passing solution of impure geldanamycin through a bed of adsorbent preferably alumina and collecting the flow-through;
c. optionally washing the bed of adsorbent from step (b) with organic solvent or a mixture of organic solvents and collecting the elute;
d. optionally combining the flow-through from step (b) and elute from step (c);
e. concentrating the combined product layer from step (d) so that the concentrate weight becomes about 10 times the weight of the product;
f. crystallizing geldanamycin optionally cooling to the temperature less than 15° C.;
g. filtrating the crystals obtained in step (f) and
h. drying

31. The process as claimed in claim 30, wherein said organic solvents are selected from a group comprising alcohols, alkanes, chlorinated alkanes, ketones, acetates or ethers.

32. The process as claimed in claim 31, wherein said alcohols are selected from a group comprising methanol, ethanol, propanol or butanol.

33. The process as claimed in claim 31, wherein said alkanes are selected from a group comprising hexane, heptane or pentane.

34. The process as claimed in claim 31, wherein said chlorinated alkanes are selected from a group comprising methylene di chloride, ethylene di chloride or chloroform.

35. The process as claimed in claim 31, wherein said ketones are selected from a group comprising acetone, methyl ethyl ketone or methyl isobutyl ketone.

36. The process as claimed in claim 31, wherein said acetates are selecting from ethyl acetate, propyl acetate or butyl acetate.

37. The process as claimed in claim 31, wherein said ethers are selecting from diethyl ether, petroleum ether or diisopropyl ether.
